(19) United States
(12) Patent Application Publication
(10) Pub. No.: US 2019/0076478 A1
(43) Pub. Date: Mar. 14, 2019

(54) CANINE BLOOD PLATELET PREPARATIONS

(71) Applicant: Cellphire, Inc., Rockville, MD (US)

(72) Inventors: Anne S. Hale, Placitas, NM (US); Rafael Jorda, Bethesda, MD (US); Keith Moskowitz, Rockville, MD (US)

(21) Appl. No.: 16/130,727

(22) Filed: Sep. 13, 2018

Related U.S. Application Data
(60) Provisional application No. 62/558,050, filed on Sep. 13, 2017.
(60) Provisional application No. 62/684,008, filed on Jun. 12, 2018.

(51) Int. Cl. A61K 35/19; A01N 1/02; A61K 35/16; A61D 7/00

(52) U.S. Cl. CPC A61K 35/19; A01N 1/02; A01K 2227/10; A61D 7/00; A61K 35/16

(57) ABSTRACT

The present disclosure provides dry and liquid compositions that include canine platelets and/or canine platelet-derived substances in dried form or rehydrated form from a dried form of canine platelets and/or canine platelet-derived substances, as well as processes for preparing such compositions. The disclosure also provides processes for making the compositions and methods of using the compositions for therapeutic, prophylactic, diagnostic, and research purposes, and kits comprising the compositions.

### CROSS-REFERENCE TO RELATED APPLICATIONS

This application claims priority to U.S. Provisional Application No. 62/558,050, filed Sep. 13, 2017 and 62/684,008, filed Jun. 12, 2018, each of which are incorporated by reference in its entirety herein.

### STATEMENT OF GOVERNMENT INTEREST

This invention was made with government support under contract number HHS0100201300021C, awarded by Biomedical Advanced Research and Development Authority (BARDA) of the U.S. Department of Health and Human Services. The government has certain rights in the invention.

### BACKGROUND

### Technical Field

The present disclosure relates to the field of blood and blood products. More specifically, it relates to canine platelets and platelet compositions, including those containing stabilized dried platelets or compositions derived from canine platelets.

### Description of Related Art

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Unactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors. The normal canine circulating platelet count is between 175,000 and 500,000 per microliter (μl) of blood.

When bleeding from an injured blood vessel occurs, platelets gather at the site of injury by binding to exposed collagen on endothelial cells, and block the out-flow of blood from the injured blood vessel through the process of hemostasis, which results in coagulation. Coagulation is a complex process involving platelets and multiple proteins circulating in the blood system. Further, platelets contain a number of important growth factors within their alpha granules that contribute to the process of hemostasis, coagulation, and ultimately wound healing. Studies have found that growth factors, such as platelet derived wound healing factors (PDWHF), platelet-derived growth factor (PDGF), transforming growth factor (TGF), and insulin growth factors (IGF), among others, are important in different stages of the wound healing cascade and greatly influence mitogenic and cellular differentiation activities.

As discussed above, a critical function of the blood clotting system is to stop blood loss from injured tissues, such as tissues that have been damaged by injury, wounds, surgery, or other trauma. However, sometimes the wound or trauma is so great that the blood system of the injured subject is unable to rapidly and effectively stop all of the bleeding. Furthermore, while hemostasis is provided satisfactorily in most subjects, in some subjects, hemostasis is impaired such that adequate clotting is not provided, and extensive, sometimes deadly, bleeding occurs as a result of injury, wounds, surgery, or other trauma. Thus, there are often times when a subject is in need of additional platelets or platelet-derived material to provide the clotting function that is missing or inadequate.

In addition to their use “as is” to supply blood clotting functions to subjects in need, platelets, including canine platelets, are studied extensively in the laboratory to characterize their properties and understand their precise role in the blood clotting cascade. Research on platelets provides information on blood clotting factors that are supplied by the platelets, factors that interact with the platelets to promote clotting and wound healing, and factors that are necessary to activate platelets or otherwise attract platelets to, and retain them at, a site of injury.

Both the therapeutic and research uses for platelets require that platelets, or compositions derived from platelets, be available in a form that is biologically active. Currently, platelets for therapeutic uses (e.g., infusion for hemostasis) are typically provided as freshly isolated products, which are less than five days old, and for canines, preferably no more than three days old. As can be immediately recognized, maintaining an adequate supply of fresh platelets for use in subjects in need is costly and results in loss of a large amount of platelets due to expiration prior to use, particularly in rural settings and combat theaters. Furthermore, because fresh platelets are so important for use in therapy, it can be difficult and expensive to obtain fresh platelets for research purposes. Thus, there is a need in the veterinary art for alternatives to fresh platelets for therapy and research.

Even though numerous advances in blood products and wound healing have taken place over the last several years, there is still a need for improved compositions for treating wounds by hemostasis and treating coagulopathy. There is accordingly a need for improved methods of making compositions for treating wounds and/or coagulopathy. Likewise, there is a need for methods for treating wounds to stop blood loss that are rapid, effective, and suitable for use in multiple settings.

### SUMMARY

The present disclosure addresses needs in the veterinary art by providing hemostatic compositions derived from canine platelets. The hemostatic compositions are prepared by a process that includes loading the platelets with a cryoprotectant and drying the platelets under controlled conditions. In embodiments, the process for preparation of the hemostatic compositions further includes rehydrating (also referred to in the art as reconstituting) the hemostatic compositions. The hemostatic compositions can be used for numerous purposes, including, but not limited to, use as hemostatic agents to form clots at sites of injury involving bleeding, use for treating coagulopathy, and use to promote tissue regeneration and healing. The present disclosure also provides compositions and methods for preventing or treating expected or active excessive bleeding associated with anticoagulant therapy or other therapies or environmental effects that result in inhibition of the clotting cascade. The present disclosure also addresses needs in the art by providing compositions and methods that can be used as diagnostics for detection of blood clotting disorders. Accordingly, the present disclosure provides methods for making diagnostic compositions and using them in methods for diagnosing bleeding disorders. The present disclosure further addresses needs in the veterinary art by providing methods for preparing dried canine hemostatic compositions, and reconstituted hemostatic compositions. Methods of this disclosure provide dried canine platelets that are stable for extended periods of time at a wide range of temperatures. The methods, and the compositions, also provide dried hemostatic products that, upon reconstitution, function well in the process of blood clotting, and thus can be used successfully in therapeutic applications, such as for wound healing and treatment of bleeding diseases and disorders. Kits are provided to contain the compositions.

### DESCRIPTION OF DRAWINGS

FIGS. 1A-1B shows flow cytometry data of an exemplary composition comprising lyophilized canine platelets in a histogram plot (FIG. 1A) and a density plot (FIG. 1B), respectively, to detect observable reactivity to a human clone of antibody CD41.

FIGS. 2A-2B shows flow cytometry data of an exemplary composition comprising lyophilized canine platelets in a histogram plot (FIG. 2A) and a density plot (FIG. 2B), respectively, to detect observable reactivity to a human clone of antibody CD61.

FIGS. 3A-3B shows flow cytometry data of an exemplary composition comprising lyophilized canine platelets in a histogram plot (FIG. 3A) and a density plot (FIG. 3B), respectively, to detect observable reactivity to a human close of antibody CD42.

FIGS. 4A-4B shows flow cytometry data of an exemplary composition comprising lyophilized canine platelets in a histogram plot (FIG. 3A) and a density plot (FIG. 3B), respectively, to detect observable reactivity to a human clone of antibody CD9.

FIG. 5 shows flow cytometry data of an exemplary composition in a density plot to detect observable reactivity to antibodies CD41 and CD61.

FIG. 6 shows flow cytometry data of an exemplary composition in a stacked density plot to detect observable reactivity to antibodies CD42 and CD9.

FIG. 7 shows comparative particle size distribution data of two exemplary hemostatic compositions processed under different pH maintenance conditions (Series 1=pH 5.43; Series 2=pH 6.2).

FIGS. 8A and 8B shows flow cytometry dot plots for exemplary compositions processed in different types of closed containers. FIG. 8A provides the data of compositions processed in bottle containers (Group X) and FIG. 8B provides the data of compositions processed in bags (Group Y).

FIG. 9 shows a graph of blood loss averages in canine test subjects treated with varying doses of exemplary compositions.

FIG. 10 shows blood assessment (DOGiBAT) comparative data of an exemplary hemostatic composition (StablePlate Rx®) and DMSO cryopreserved platelets.

### DETAILED DESCRIPTION

Reference will now be made in detail to various exemplary embodiments provided herein. It is to be understood that the following discussion of exemplary embodiments is not intended as a limitation on this disclosure, as broadly disclosed herein. Rather, the following discussion is provided to give the reader a more detailed understanding of certain aspects and features of this disclosure.

Before embodiments of the present disclosure are described in detail, it is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9,1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “canine”, “patient”, “individual” and other terms used in the art to indicate one who is subject to a veterinary treatment. In addition, the use of the term “canine” is to be understood to include all species, subspecies, and breeds of the genus Canis, including domesticated house dogs and military or police dogs.

In one aspect of this disclosure, a hemostatic composition derived from canine platelets is provided. The hemostatic composition can comprise dried canine platelets, dried particles derived from canine platelets, or a combination of the two. Alternatively, the composition can comprise rehydrated dried canine platelets, rehydrated dried particles derived from canine platelets, or a combination of the two. As such, the hemostatic composition can be in either dry form or liquid form. When in dry form, the hemostatic composition contains less than ten (10) percent (<10%), preferably less than five percent (<5%), and more preferably less than two percent (<2%) residual moisture. When in liquid form, the liquid portion of the composition can be water, an aqueous liquid, blood or a blood component or fraction (such as plasma), saline, buffered saline (e.g., phosphate buffered saline), or the like.

The hemostatic composition is preferably sterile and has less than two Endotoxin Units (EU) per milliliter (ml) when in liquid form. The liquid form of the hemostatic composition has 50% or more, preferably 70% or more ofparticles in the range of 0.5 μm to 0.9 μm, whereas greater than 95% of freshly isolated, normal, resting (unactivated) canine platelets show a size range of 1 μm to 3 μm. (Wilkerson et al., 2001). In some embodiments, the hemostatic composition (e.g., dry or liquid hemostatic compositions) does not contain DMSO. It further does not have crosslinking of platelet membranes via proteins and lipids present on the membranes. In some embodiments the hemostatic composition (e.g., dry or liquid hemostatic compositions) has less than about 10%, such as less than about 8%, such as less than about 6%, such as less than about 4%, such as less than about 2%, such as less than about 0.5% crosslinking of platelet membranes via proteins and/or lipids present on the membranes. A canine hemostatic composition ofthe present disclosure is thus physically distinct from a fresh canine platelet composition or other rehydrated lyophilized platelets known in the art.

In some embodiments, the hemostatic composition provided herein includes platelets and/or platelet-derived particles having a particle size (e.g., diameter, max dimension) of at least about 0.2 μm (e.g., at least about 0.3 μm, at least about 0.4 μm, at least about 0.5 μm, at least about 0.6 μm, at least about 0.7 μm, at least about 0.8 μm, at least about 0.9 μm, at least about 1.0 μm, at least about 1.0 μm, at least about 1.5 μm, at least about 2.0 μm, at least about 2.5 μm, or at least about 5.0 μm). In some embodiments, the hemostatic composition provided herein includes particles having a particle size of less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the hemostatic composition provided herein includes particles having a particle size of from about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, the hemostatic composition has at least 50% (e.g., at least about 55%, at least about 60%, at least about 65%, at least about 70%, at least about 75%, at least about 80%, at least about 85%, at least about 90%, at least about 95%, or at least about 99%) of platelets and/or platelet-derived particles in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 um to about 1.0 μm, from about 0.5 μm to about 0.9 μm, or from about 0.6 μm to about 0.8 μm). In some embodiments, the hemostatic composition has at most 99% (e.g., at most about 95%, at most about 80%, at most about 75%, at most about 70%, at most about 65%, at most about 60%, at most about 55%, or at most about 50%) of platelets and/or platelet-derived particles in the range of about 0.3 μm to about 5.0 um (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, the hemostatic composition has about 50% to about 99% (e.g., about 55% to about 95%, about 60% to about 90%, about 65% to about 85, about 70% to about 80%) of platelets and/or platelet-derived particles in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 um, from about 0.5 μm to about 0.9 μm, or from about 0.6 μm to about 0.8 μm).

In some embodiments, the particle count in the composition is from about 1.3×10⁹/mL to about 2.1×10⁹/mL.

The composition retains a sufficient level of components necessary for the blood clotting function of platelets when introduced into subjects in need of platelet functions. The composition can comprise other blood components, and in particular can comprise blood clotting factors, such as Factor VII and Factor VIII, in their normal or activated states. These other components may be present as a result of concentrating of the platelets or they may be added as separately purified components to the platelets prior to or after drying (e.g., during the rehydration period). These other blood components may be present singly (i.e., only one is present in the composition), or multiple other blood components may be included in the composition together with the platelets and/or platelet-derived particles. Typically, the other blood components are included in amounts or concentrations that, when administered to a subject, provide a detectable change in at least one physiological process of the treated subject, or provide a known benefit.

Additionally, the hemostatic composition of the present disclosure does not show observable reactivity to a human clone of an antibody that binds to CD42 when assayed by fluorescence in a Gallios flow cytometer running Gallios software Version 1.2. In some embodiments, the hemostatic composition of the present disclosure does not show observable reactivity to a human clone of an antibody that binds to CD42b when assayed by fluorescence in a Gallios flow cytometer running Gallios software Version 1.2. Conversely, the hemostatic composition of the present disclosure does show observable reactivity to a human antibody that binds to CD61, a human clone of an antibody that binds to CD41, and a human antibody that binds to CD9 when assayed by fluorescence in a Gallios flow cytometer running Gallios software Version 1.2.

A hemostatic composition of this disclosure has, when in liquid form, a pH of greater than 5.0, preferably above 5.5, and more preferably in a pH range of 6.4 to 7.4, during the process of preparation and upon rehydration. Further, it is preferred that the liquid form of the composition has a lactate concentration of less than 2.5 mmol/L.

In some embodiments a hemostatic composition of this disclosure has, when in liquid form, a pH of greater than about 5.0, such as above about 5.5, such as in a pH range of about 6.4 to about 7.4, during the process of preparation and upon rehydration.

In some embodiments a hemostatic composition of this disclosure has a pH lower than about 10.0, such as lower than about 9.0, such as lower than about 8.0, such as lower than about 7.5.

Further, it is preferred that the liquid form of the composition has a lactate concentration of less than about 11 mmol/L, such as less than about 10 mmol/L, such as less than about 9 mmol/L, such as less than about 8 mmol/L.

A hemostatic composition of this disclosure can also comprise additional biologically active or biologically inactive components. For example, the composition can comprise some or all of the additional components discussed below with regard to the process of making the hemostatic composition, such as, but not limited to, salts, buffers a cryoprotectant, sugars, and a lyoprotectant.

In another aspect, this disclosure provides a process for making the hemostatic composition provided herein. In some embodiments, the process includes a first step of obtaining a liquid composition that comprises canine platelets. In some embodiments, the process includes a first step of providing a composition that comprises canine platelets and water. In embodiments, the process can include purifying the platelets to a desired extent, for example to form platelet rich plasma (PRP). The step of purifying the platelets can use any method known in the art as useful for obtaining purified platelets, including centrifugation (such as differential centrifugation) and filtration. Alternatively, plateletpheresis can be used to provide PRP.

The process further includes incubating the platelets in a solution that includes a cryoprotectant (e.g., a non-reducing disaccharide) for a sufficient amount of time and at a suitable temperature to allow for entry of the cryoprotectant into the platelets (also referred to herein as “loading” the platelets). The cryoprotectant is thought to stabilize proteins and other biological substances in the interior of the platelets. The identity of the cryoprotectant is not limited as long as it can enter the platelets and provide a cryoprotectant property. Non-limiting examples of suitable cryoprotectants are saccharides, such as monosaccharides and disaccharides, including sucrose, maltose, trehalose, glucose, mannose, and xylose.

A preferred saccharide for use in the process of preparing a hemostatic composition provided herein is trehalose. Regardless of the identity of the saccharide, it can be present in the composition in any suitable amount. For example, it can be present in an amount of 1 mM to 1M. In embodiments, it is present in an amount of from 10 mM 10 to 500 mM. In some embodiments, it is present in an amount of from 20 mM to 200 mM. In embodiments, it is present in an amount from 40 mM to 100 mM. Of course, in various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

In another embodiment, provided herein is a hemostatic composition obtained by a process comprising the steps of:

providing a composition comprising canine platelets in a gas-permeable container;

adding a cryoprotectant to the composition;

incubating the canine platelets in the composition;

adding a lyoprotectant to the composition; and

drying the composition;

wherein the pH of the composition during the incubating, the drying, or both, is greater than 5.0.

In another embodiment, provided herein is a hemostatic composition obtained by a process comprising the steps of:

incubating a liquid composition that comprises canine platelets in a solution that includes a cryoprotectant;

adding a lyoprotectant to form a mixture; and

drying the mixture;

wherein the process includes maintaining the pH above 5.

In another embodiment, provided herein is a hemostatic composition obtained by a process comprising the steps of:

providing in a gas-permeable container a first composition comprising canine platelets and a solvent, such as water;

incubating in the gas-permeable container the first composition with a cryoprotectant to form a second composition;

adding a lyoprotectant to the second composition to form a third composition; and

drying the third composition to form a fourth composition;

wherein the pH of one or more of the first composition, the second composition, and the third composition, is greater than 5.0.

In another embodiment, provided herein is a hemostatic composition obtained by a process comprising the steps of:

providing in a gas-permeable container a first composition comprising canine platelets, a solvent, such as water, and a lyoprotectant; incubating in the gas-permeable container the first composition with a cryoprotectant to form a second composition; and

drying the second composition to form a third composition;

wherein the pH of one or more of the first composition and the second composition is greater than 5.0.

In another embodiment, provided herein is a process for preparing a hemostatic composition, the process comprising the steps of:

providing a composition comprising canine platelets in a gas-permeable container;

adding a cryoprotectant to the composition;

incubating the canine platelets in the composition;

adding a lyoprotectant to the composition; and

drying the composition;

wherein the pH of the composition during the incubating, the drying, or both, is greater than 5.0.

In another embodiment, provided herein is a process for preparing a hemostatic composition, the process comprising the steps of:

incubating a liquid composition that comprises canine platelets in a solution that includes a cryoprotectant;

adding a lyoprotectant to form a mixture; and

drying the mixture;

wherein the process includes maintaining the pH above 5.

In another embodiment, provided herein is a process for preparing a hemostatic composition, the process comprising the steps of:

providing in a gas-permeable container a first composition comprising canine platelets and a solvent, such as water;

incubating in the gas-permeable container the first composition with a cryoprotectant to form a second composition;

adding a lyoprotectant to the second composition to form a third composition; and

drying the third composition to form a fourth composition;

wherein the pH of one or more of the first composition, the second composition, and the third composition, is greater than 5.0.

In another embodiment, provided herein is process for preparing a hemostatic composition, the process comprising the steps of:

providing in a gas-permeable container a first composition comprising canine platelets, a solvent, such as water; and a lyoprotectant; incubating in the gas-permeable container the first composition with a cryoprotectant to form a second composition; and

drying the second composition to form a third composition;

wherein the pH of one or more of the first composition and the second composition is greater than 5.0.

In some of the embodiments wherein a hemostatic composition is obtained by a process disclosed herein, the hemostatic composition is the composition obtained from the drying step.

In some of the embodiments wherein a hemostatic composition is obtained by a process disclosed herein, the composition obtained from the drying step is rehydrated to form the hemostatic composition. Thus, in such embodiments, the process further comprises rehydrating the composition obtained from the drying step, to form the hemostatic composition.

The step of incubating the platelets to load them with a cryoprotectant includes incubating the platelets for a time suitable for loading, as long as the time, taken in conjunction with the temperature, is sufficient for the cryoprotectant to come into contact with the platelets and, preferably, be incorporated, at least to some extent, into the platelets. In embodiments, incubation is carried out for about 1 minute to about 180 minutes or longer.

The step of incubating the platelets to load them with a cryoprotectant includes incubating the platelets and the cryoprotectant at a temperature that, when selected in conjunction with the amount of time allotted for loading, is suitable for loading. In general, the composition is incubated at a temperature above freezing for at least a sufficient time for the cryoprotectant to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

The process for making the hemostatic composition provided herein can include incubating the canine platelets in an aqueous solution that is buffered. The buffering component may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TB S). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES).

It has been surprisingly found that, during the process of making hemostatic compositions provided herein, the pH of the loading solution can change substantially. As such, the present process includes monitoring and, if necessary, adjusting the pH to maintain it above 5.0, preferably above 5.5, and more preferably in the range of 6.4 to 7.4. Monitoring can be performed at any suitable time, but no less often than between each step of the process. To improve maintenance of the pH of the composition at an acceptable level, loading can be accomplished using O₂ and CO₂-permeable incubation chambers, such as certain plastic lyophilization bags (which may also be referred to incubation bags), which are commercially available (e.g., Saint-Gobain VueLife “C” Series Bags manufactured by Saint Gobain). Monitoring of the pH throughout the process provides, along with the other steps, a highly controlled process. It can be highly advantageous to maintain the pH of the composition as described above to reduce or prevent platelet aggregation in the hemostatic composition during processing. Platelet aggregation can reduce clotting function effectiveness of the hemostatic composition, thus it is desirable to prevent or reduce such aggregation from occurring.

Certain embodiments of the process provided herein can include containing the compositions provided herein in a bag (e.g., lyophilization bag) during one or more processing steps, e.g., during lyophilization of the composition provided herein. Any of the compositions provided herein can be contained in the bag during the occurrence of any one or more processes provided herein. For example, the composition may be contained in the bag during incubation, lyophilization, post-drying processing, storage, or any combinations thereof. Use of the bag is highly advantageous because it can provide flexible, transparent, chemically-resistant, biologically-resistant, heat-resistant, water-permeable, and/or gas-permeable containment during the processing and/or storage of the compositions provided herein.

In various embodiments, the bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 mL/cm² (e.g., at least about 2.1 mL/cm², at least about 2.2 mL/cm², at least about 2.3 mL/cm², at least about 2.4 mL/cm², at least about 2.5 mL/cm², at least about 2.6 mL/cm², at least about 2.7 mL/cm², at least about 2.8 mL/cm², at least about 2.9 mL/cm², at least about 3.0 mL/cm², at least about 3.1 mL/cm², at least about 3.2 mL/cm², at least about 3.3 mL/cm², at least about 3.4 mL/cm², at least about 3.5 mL/cm², at least about 3.6 mL/cm², at least about 3.7 mL/cm², at least about 3.8 mL/cm², at least about 3.9 mL/cm², at least about 4.0 mL/cm², at least about 4.1 mL/cm², at least about 4.2 mL/cm², at least about 4.3 mL/cm², at least about 4.4 mL/cm², at least about 4.5 mL/cm², at least about 4.6 mL/cm², at least about 4.7 mL/cm², at least about 4.8 mL/cm², at least about 4.9 mL/cm², or at least about 5.0 mL/cm². In some embodiments, the SA/V ratio of the container can be at most about 10.0 mL/cm² (e.g., at most about 9.9 mL/cm², at most about 9.8 mL/cm², at most about 9.7 mL/cm², at most about 9.6 mL/cm², at most about 9.5 mL/cm², at most about 9.4 mL/cm², at most about 9.3 mL/cm², at most about 9.2 mL/cm², at most about 9.1 mL/cm², at most about 9.0 mL/cm², at most about 8.9 mL/cm², at most about 8.8 mL/cm², at most about 8.7 mL/cm², at most about 8.6 , mL/cm² at most about 8.5 mL/cm², at most about 8.4 mL/cm², at most about 8.3 mL/cm², at most about 8.2 mL/cm², at most about 8.1 mL/cm², at most about 8.0 mL/cm², at most about 7.9 mL/cm², at most about 7.8 mL/cm², at most about 7.7 mL/cm², at most about 7.6 mL/cm², at most about 7.5 mL/cm², at most about 7.4 mL/cm², at most about 7.3 mL/cm², at most about 7.2 mL/cm², at most about 7.1 mL/cm², at most about 6.9 mL/cm², at most about 6.8 mL/cm², at most about 6.7 mL/cm², at most about 6.6 mL/cm², at most about 6.5 mL/cm², at most about 6.4 mL/cm², at most about 6.3 mL/cm², at most about 6.2 mL/cm², at most about 6.1 mL/cm², at most about 6.0 mL/cm², at most about 5.9 mL/cm², at most about 5.8 mL/cm², at most about 5.7 mL/cm², at most about 5.6 mL/cm², at most about 5.5 mL/cm², at most about 5.4 mL/cm², at most about 5.3 mL/cm², at most about 5.2 mL/cm², at most about 5.1 mL/cm², at most about 5.0 mL/cm², at most about 4.9 mL/cm², at most about 4.8 mL/cm², at most about 4.7 mL/cm², at most about 4.6 mL/cm², at most about 4.5 mL/cm², at most about 4.4 mL/cm², at most about 4.3 mL/cm², at most about 4.2 mL/cm², at most about 4.1 mL/cm², or at most about 4.0 mL/cm². In some embodiments, the SA/V ratio of the container can range from about 2.0 to about 10.0 mL/cm² (e.g., from about 2.1 mL/cm²to about 9.9 mL/cm², from about 2.2 mL/cm² to about 9.8 mL/cm², from about 2.3 mL/cm² to about 9.7 mL/cm², from about 2.4 mL/cm² to about 9.6 mL/cm², from about 2.5 mL/cm² to about 9.5 mL/cm², from about 2.6 mL/cm² to about 9.4 mL/cm², from about 2.7 mL/cm² to about 9.3 mL/cm², from about 2.8 mL/cm² to about 9.2 mL/cm², from about 2.9 mL/cm² to about 9.1 mL/cm², from about 3.0 mL/cm² to about 9.0 mL/cm², from about 3.1 mL/cm² to about 8.9 mL/cm², from about 3.2 mL/cm² to about 8.8 mL/cm², from about 3.3 mL/cm² to about 8.7 mL/cm², from about 3.4 mL/cm² to about 8.6 mL/cm², from about 3.5 mL/cm² to about 8.5 mL/cm², from about 3.6 mL/cm² to about 8.4 mL/cm², from about 3.7 mL/cm² to about 8.3 mL/cm², from about 3.8 mL/cm² to about 8.2 mL/cm², from about 3.9 mL/cm² to about 8.1 mL/cm², from about 4.0 mL/cm² to about 8.0 mL/cm², from about 4.1 mL/cm² to about 7.9 mL/cm², from about 4.2 mL/cm² to about 7.8 mL/cm², from about 4.3 mL/cm² to about 7.7 mL/cm², from about 4.4 mL/cm² to about 7.6 mL/cm², from about 4.5 mL/cm² to about 7.5 mL/cm², from about 4.6 mL/cm² to about 7.4 mL/cm², from about 4.7 mL/cm² to about 7.3 mL/cm², from about 4.8 mL/cm² to about 7.2 mL/cm², from about 4.9 mL/cm² to about 7.1 mL/cm², from about 5.0 mL/cm² to about 6.9 mL/cm², from about 5.1 mL/cm² to about 6.8 mL/cm², from about 5.2 mL/cm² to about 6.7 mL/cm², from about 5.3 mL/cm² to about 6.6 mL/cm², from about 5.4 mL/cm² to about 6.5 mL/cm², from about 5.5 mL/cm² to about 6.4 mL/cm², from about 5.6 mL/cm² to about 6.3 mL/cm², from about 5.7 mL/cm² to about 6.2 mL/cm², or from about 5.8 mL/cm² to about 6.1 mL/cm².

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

The process of preparing compositions provided herein can also comprise adding to the loading solution one or more salts, such as phosphate salts, sodium salts, potassium salts, calcium salts, magnesium salts, and any other salt that can be found in blood or blood products, or that is known to be useful in drying platelets, or any combination of two or more of these. Preferably, these salts are present in the composition at an amount that is about the same as is found in whole blood.

The process of preparing a dried canine platelet hemostatic composition includes introducing a lyoprotectant, such as a high molecular weight polymer, into the loading composition. By “high molecular weight” it is meant a polymer having an average molecular weight of about or above 70 kDa. Non-limiting examples are polymers of sucrose and epichlorohydrin, such as those sold under the trade names Ficoll® 70 and Ficoll® 400 (GE Healthcare Bioprocess, Uppsala, Sweden). Although any amount of high molecular weight polymer can be used, it is preferred that an amount be used that achieves a final concentration of about 3% to 10% (w/v), such as 3% to 7%, for example 6%. Other non-limiting examples of lyoprotectants are serum albumin, dextran, polyvinyl pyrolidone (PVP), starch, and hydroxyethyl starch (HES).

In some embodiments, the process for preparing a composition includes adding an organic solvent, such as the alcohol ethanol, to the loading solution. In such a loading solution, the solvent can range from 0.1% to 5.0% (v/v).

Within the process provided herein for making a dried canine platelet hemostatic composition, addition of the lyoprotectant can be the last step prior to drying. However, in some embodiments, the lyoprotectant is added at the same time or before the cryoprotectant or other components of the loading composition. Preferably, the lyoprotectant is added to the loading solution, thoroughly mixed to form a drying solution, dispensed into a drying vessel (e.g., a glass or plastic serum vial, a lyophilization bag), and subjected to conditions that allow for drying of the solution to form a dried canine platelet-derived hemostatic composition.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table 1. Additional exemplary lyophilization methods can be found in U.S. Pat. No. 7,811,558, U.S. Pat. No. 8,486,617, and U.S. Pat. No. 8,097,403. An exemplary spray-drying method includes: combining nitrogen, as a drying gas, with a loading solution according to the present disclosure, then introducing the mixture into GEA Mobile Minor spray dryer from GEA Processing Engineering, Inc. (Columbia, Md., USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of10 to 35 minutes. The final step in spray drying is preferentially collecting the dried mixture. The dried canine platelet-derived hemostatic composition of the present disclosure is stable for at least six months at temperatures that range from −20° C. or lower to 90° C. or higher.

TABLE 1
Exemplary Lyophilization Protocol

| | Step | Temp. Set | | Type | Duration | | Pressure Set |
|---|---|---|---|---|---|---|---|
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | var | | N/A |
| Primary Dry | P1 | −40° | | Hold | 1.5 | Hrs | 0 mT |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In embodiments relating to dried compositions, the compositions can be heated, such as in the range of about 30° C. to about 90° C., such as about 20° C. to about 40° C., including without prejudice, 37° C. The heating process can promote formation of platelet-derived compositions that are suitable for use in methods of treatment and for use in assays of platelet function. The heating process further can improve stability. In the embodiments that include a post-drying heating step, the dried compositions can be heated from less than one minute up to 24 hours or more. Typically, heating is conducted from about 14 hours to about 24 hours.

The dried compositions provided herein are highly stable, having a shelf-life of at least six months or above, such as at least eighteen months, at room temperature or below. For example, the dried compositions, when rehydrated, can show hemostatic triggers for primary coagulation properties up to one year after manufacture when stored at room temperature or below, up to 18 months at room temperature or below, or even longer. By “stable” it is meant that the platelets of the compositions, when rehydrated, (i.e., liquid compositions, as discussed below) function within the parameters mentioned above, and provide adequate hemostatic triggers for primary coagulation. These clotting functions include hemostatic function and primary coagulopathic function. This stability is of great advantage in providing platelet products to those in need, particularly those found at sites some distance from blood collection centers, those in combat theaters, and those working in disaster areas as first responders. Furthermore, because the compositions can be stored at room temperature up to 40° C. for long-term storage and up to 80° C. to 90° C. for short periods of about 24 hours, complicated, bulky, or expensive containers for storage (e.g., refrigerators) are not needed. That is, the problem of cold-chain storage is eliminated.

When needed for treatment of bleeding and for formation of clots, use as a primary hemostatic agent, or for research purposes, the dried compositions of this disclosure can be rehydrated. The rehydrated compositions are considered liquid compositions according to this disclosure. The dried compositions are preferably rehydrated with water (preferably sterile) or another aqueous liquid, which can, but does not necessarily, include a buffering component. Preferably, the amount of liquid used to rehydrate the dried compositions is an amount that provides a concentration of composition components that is about the same osmolality as canine blood. Those of skill in the art can adjust the amount of liquid used to form the loading and drying solutions, and to rehydrate the dried composition, to achieve a suitable platelet function level and osmolality without undue or excessive experimentation. Liquid compositions of this disclosure typically have a shelf-life of a few hours or less. Consistent with the disclosure above with respect to preparation of dried compositions, the pH of the liquid compositions should be monitored at pre-selected time points and adjusted, if necessary, to maintain a suitable pH.

As will be recognized by those of skill in the art, the composition of this disclosure has the ability to act as a hemostatic agent to form clots at sites of injury involving bleeding. This concept can be understood as use of the composition in a method for treating bleeding or a method for treating a subject having a bleeding wound. In general, the method comprises contacting a site of bleeding with a sufficient amount of a composition provided herein to reduce or stop the bleeding. The step of contacting can be performed in any suitable way, including, but not necessarily limited to, i) systemic administration of the composition via intravenous infusion or bolus injection and ii) topical administration directly to the site of bleeding. For intravenous administration, the composition is a liquid composition. It should be noted that intravenous administration is suitable for both treating bleeding due to a wound or other trauma and treating bleeding due to coagulopathy. For topical administration, the composition can be either liquid or dry. When topically administering the liquid form, the composition can be dripped, sprayed, or poured (or the equivalent) directly onto the site of bleeding. When topically administering the dry form, the composition can be sprinkled or sprayed (or the equivalent) directly onto the site of bleeding, or directly administered to the site of bleeding as part of a bandage or dressing. The methods of treating, whether therapeutic or prophylactic, of this disclosure can further comprise administering a composition provided herein a second or multiple times. Therefore, the methods of this disclosure encompass treatment regimens in which administration is repeated one or more times at preselected time intervals. Successive administrations may include administration of additional components. The choice of amounts and composition components can be selected by those of skill in the art based on various parameters commonly considered by those of skill in the art, such as subject age, weight, health history, clinical presentation, ancillary presentations, and the like. It is well within the skill of those in the art to make appropriate changes and adjustments to treatment regimens without undue experimentation.

As should be evident, this disclosure t provides dry and liquid canine hemostatic compositions for the treatment of drug-induced coagulopathy and for the accelerated efficacy of procoagulant drugs. For example, the compositions of the invention overcome the deficiencies seen in anticoagulant therapy subjects and other subjects showing delayed or absent clotting by providing at least one component in the clotting cascade that is downstream of the component that is lacking in these subjects.

Viewed in another way, the invention comprises administering the composition of the invention to a subject in an amount sufficient to raise the hemostatic or coagulation properties of that subject's blood to a level that is detectably higher than it was before administration. The method can further comprise administering other biologically active agents, such as clotting factors and/or chemotherapeutic agents for treatment of cancer.

In yet an additional aspect, the invention provides methods of monitoring the progression of a disease or disorder of the blood clotting system. The methods generally comprise combining a composition provided herein with platelets and/or plasma removed from a subject suffering from the disease or disorder to make a mixture, and determining the blood clotting ability of the mixture. Typically, determining the blood clotting ability of the mixture indicates the blood clotting ability of the subject's blood, and comprises assaying clotting time of the mixture. Furthermore, typically, multiple assays are performed over time to give an indication of progression over time.

A further aspect of this disclosure provides kits. In general, a kit of this disclosure comprises a composition provided herein. The kits of this disclosure typically comprise at least one container containing a composition provided herein, and can further comprise optional components, such as sterile aqueous liquid for rehydrating a dry composition to form a liquid composition, equipment for administering the compositions, and the like. The container can be any material suitable for containing the composition, such as a vial, an ampule, or a bag. In embodiments, the container comprises a sufficient amount of composition to perform at least one embodiment of at least one method provided herein. Thus, the kits can be, among other things, diagnostic kits, blood clotting monitoring kits for coagulation proteins or platelets, or drug treatment monitoring kits. In embodiments, the container is provided as a component of a larger kit, which includes suitable packaging and, optionally, instructions and other information relating to use of the compositions. In embodiments, the container or kit comprises other components, such as purified components of the clotting cascade. The kit can be configured to supply the composition for use in in vivo treatments, for use in in vitro diagnostics, or for use in in vitro or in vivo research. Often, the kits will comprise some or all of the supplies and reagents to perform one or more control reactions to ensure the kits are performing properly and to provide baseline results against which test samples can be compared. In embodiments, the composition is provided in a sufficient amount to treat a subject in need of platelet function, such as a subject undergoing surgery or having a bleeding wound. In other embodiments, a sufficient amount of the composition is provided to perform studies on platelets or the blood clotting system of canines.

### EXAMPLES

The following are exemplary compositions comprising lyophilized canine platelets.

### Composition A:

3.20 mg/mL NaCl
0.35 mg/mL KCl
2.01 mg/mL HEPES
0.60 mg/mL NaHCO₃
0.25 mg/mL Ethanol
0.4 mg/mL Dextrose
29.5 mg/mL Trehalose
55 mg/mL Polysucrose
Particle Count 1.89×10⁹/mL

### Composition B:

3.62 mg/mL NaCl
0.26 mg/mL KCl
2.01 mg/mL HEPES
0.60 mg/mL NaHCO₃
0.32 mg/mL Ethanol
0.48 mg/mL Dextrose
31.1 mg/mL Trehalose
62 mg/mL Polysucrose
Particle Count 1.75×10⁹/mL

### Composition C:

3.51 mg/mL NaCl
0.29 mg/mL KCl
1.81 mg/mL HEPES
0.81 mg/mL NaHCO₃
0.31 mg/mL Ethanol
0.43 mg/mL Dextrose
30.27 mg/mL Trehalose
60 mg/mL Polysucrose
Particle Count 1.66×10⁹/mL

### Composition D:

3.44 mg/mL NaCl
0.31 mg/mL KCl
1.93 mg/mL HEPES
0.72 mg/mL NaHCO₃
0.28 mg/mL Ethanol
0.51 mg/mL Dextrose
27.6 mg/mL Trehalose
65 mg/mL Polysucrose
Particle Count 1.53×10⁹/mL

### Composition E:

3.66 mg/mL NaCl
0.28 mg/mL KCl
1.89 mg/mL HEPES
0.75 mg/mL NaHCO₃
0.32 mg/mL Ethanol
0.47 mg/mL Dextrose
26.4 mg/mL Trehalose
69 mg/mL Polysucrose
Particle Count 2.11×10⁹/mL

### Composition F:

3.3 mg/mL NaCl
0.35 mg/mL KCl
1.98 mg/mL HEPES
0.7 mg/mL NaHCO₃
0.26 mg/mL Ethanol
0.52 mg/mL Dextrose
29.5 mg/mL Trehalose
62 mg/mL Polysucrose
Particle Count 1.29×10⁹/mL

As used in the exemplary compositions herein, “particle count” refers to the total count of platelets and/or platelet-derived particles.

### Example 1

### Flow Cytometry Data

An exemplary hemostatic composition (StablePlate), derived from canine platelets, was tested to determine whether the sample showed observable reactivity to various antibodies using a flow cytometry test. In particular, the composition was observed for its reactivity to antibodies CD41, CD61, CD42, and CD9 when assayed by fluorescence in a Gallios flow cytometer running Gallios software Version 1.2.

TABLE 2
StablePlate ® Surface Markers

| Antibody | Conjugate | # Total Events | # Positive Events | % Positive |
|---|---|---|---|---|
| CD41 | PE | 180000 | 150577 | 83.65% |
| CD61 | PE | 180000 | 159981 | 88.88% |
| CD42 | FITC | 180000 | 1126 | 0.63% |
| CD9 | FITC | 180000 | 155686 | 86.49% |

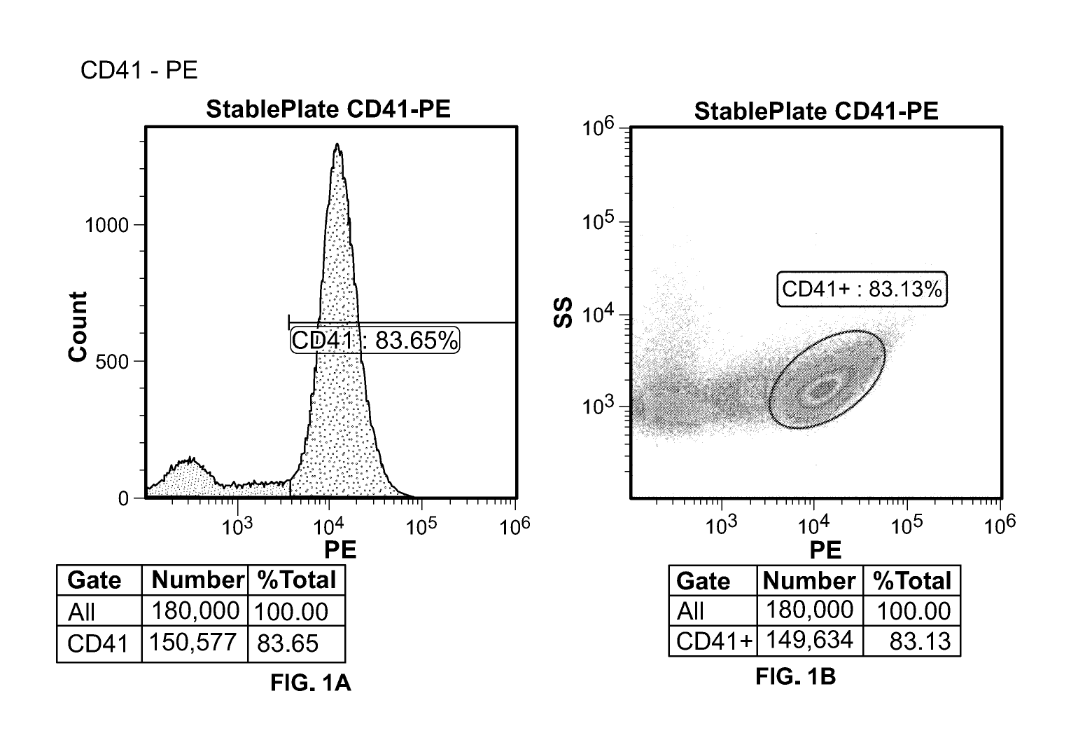

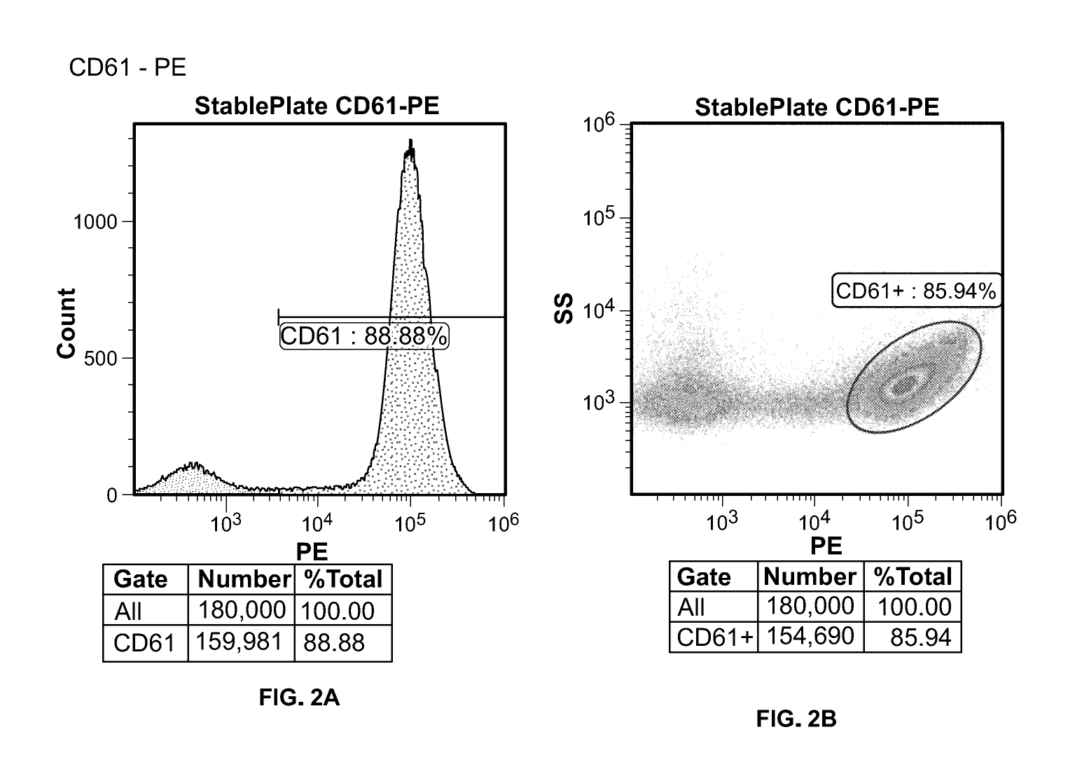

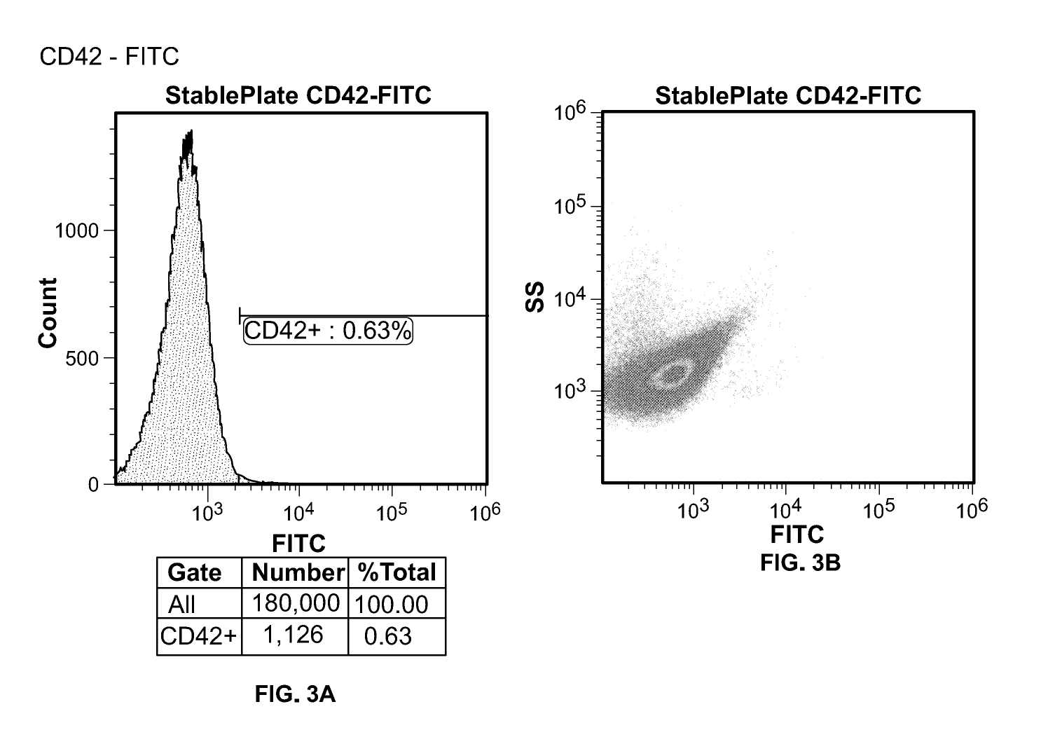

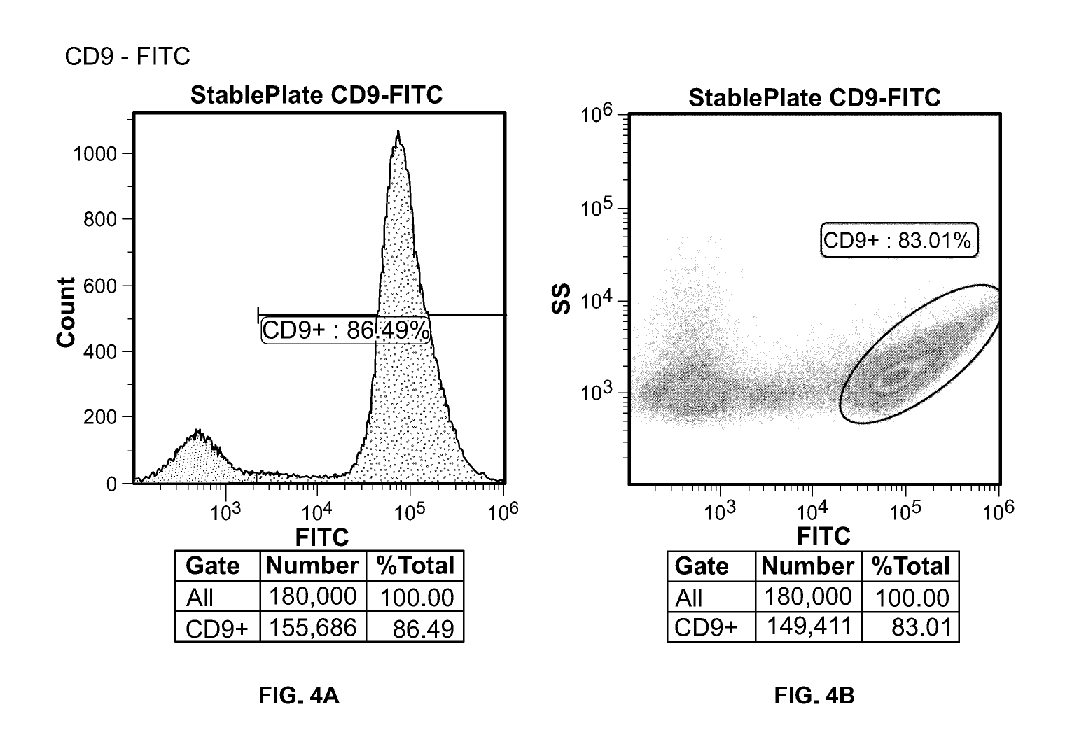

Table 2 and FIGS. 1A-1B through 4A-4B provide flow cytometry data of the exemplary composition. Specifically, FIGS. 1A-1B through 4A-4B show the flow cytometry data visually in a histogram plot (e.g., FIGS. 1A, 2A, 3A, 4A) and a density plot (e.g., FIGS. 1B, 2B, 3B, 4B). The flow cytometry test data demonstrated that Sample A had observable reactivity to human clones of antibodies CD41 (83.65% positive), CD61 (88.88% positive), CD9 (86.49% positive events), but not CD42 (0.63% positive).

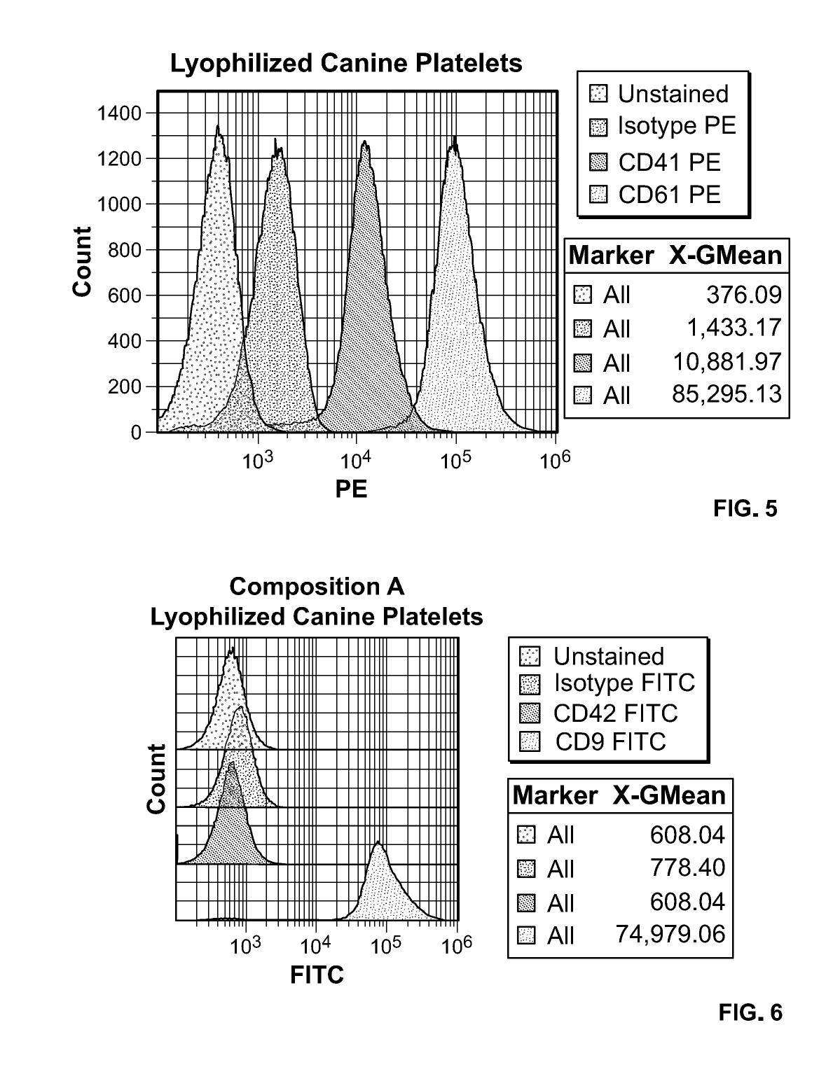

FIG. 5 shows the flow cytometry data of the composition in a density plot comparing observable reactivity to a human clone of antibodies CD41 and CD61, an unstained sample, and a non-specific isotype control using a phycoerythrin (PE) dye when assayed by fluorescence in a Gallios flow cytometer running Gallios software Version 1.2. The data showed that the composition had high particle counts of CD41 (approximately 11,000) and CD61 (approximately 85,000) relative to the non-specific isotype control count (approximately 1,400) and the unstained sample count (approximately 376). Test results demonstrated that the composition had an observable reactivity to a human clone of antibodies CD41 and CD61.

FIG. 6 shows flow cytometry data using a fluorescein isothiocyanate (FITC) fluorophore in a stacked density plot when assayed by fluorescence in a Gallios flow cytometer running Gallios software Version 1.2. The density plot compared the observable reactivity of the exemplary composition to a human clone of antibodies CD42 and CD9 to an unstained sample of the composition and a non-specific isotype control of the composition. The data showed that the composition had a high particle count of CD9 (approximately 75,000), but a low particle count of CD42 (approximately 608) relative to the non-specific isotype control (approximately 778) and the unstained sample (approximately 608). Test results demonstrated that the composition had an observable reactivity to a human clone of antibodies CD9, but not to CD42.

### Example 2

### Particle Size Distribution

Two exemplary hemostatic compositions (Compositions B1 and B2) were processed under different pH maintenance conditions. Composition B1 and Composition B2 were made under conditions in which the pH of the composition was maintained at about 5.43 and 6.2, respectively. Compositions B1 and B2 were subsequently measured for particle size distribution data.

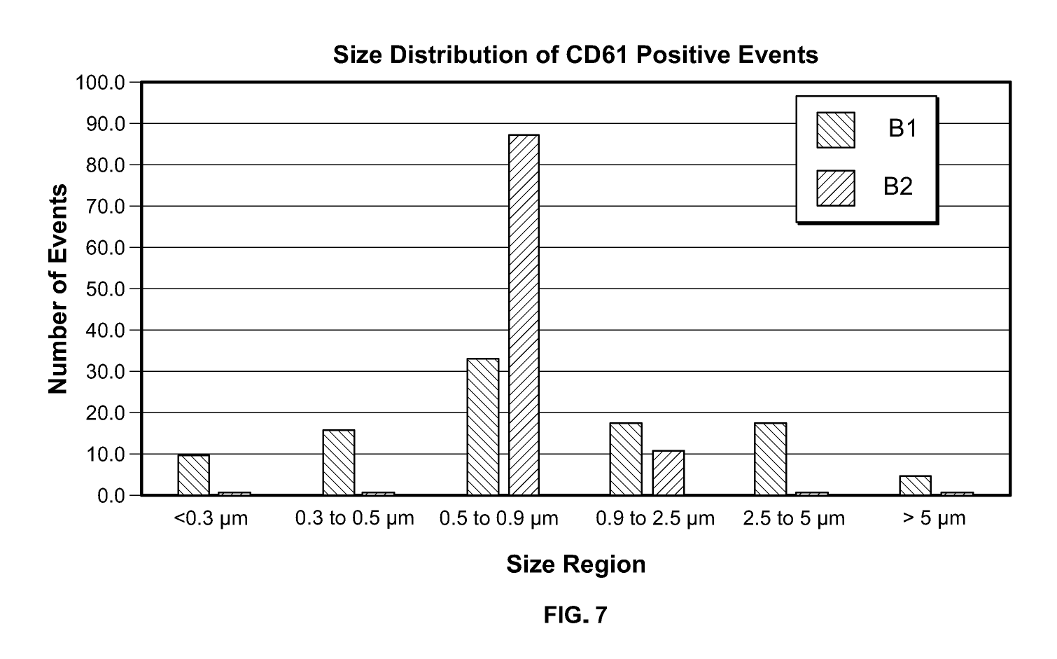

FIG. 7 provides the particle size distribution data of Compositions B1 and B2. The particle size data showed some aspects of similarity between Compositions B1 and B2, for example, the highest percentage of particles in the distribution of both B1 and B2 were within the particle size region of about 0.5-0.9 μm. Composition B2, maintained at the higher pH (6.2) condition, exhibited a tighter distribution range showing less variability while Composition B1, maintained at the lower pH (5.43) condition, had a broader particle distribution range with larger variability.

### Example 3

### pH Control and Incubation

An exemplary hemostatic composition (StablePlate Rx®) was tested at different incubation stages e.g., prior to and after different incubation conditions. The composition was tested for pH using two different methods of pH measurement, as described below, and its level of lactate.

One method of measuring pH in the composition included the use of the i-STAT® System (manufactured by Abbott Laboratories) to measure the pH level in the composition. Another method of measuring pH included the use of a standard pH meter to measure the pH level of the composition.

TABLE 3
pH Maintenance at Pre- and Post-Incubation

| | pH | pH | Lactate |
|---|---|---|---|
| | (using iStat) | (using pH meter) | (mmol/L) |
| Composition at | 6.718 | N/A | 1.98 |
| Pre-incubation | | | |
| Composition after | <6.5 | 6.16 | 8.04 |
| 1 hr incubation | | | |
| Composition after | N/A | 5.43 | 10.87 |
| 2 hr incubation | | | |

Table 3 provides the pH and lactate data obtained on the exemplary hemostatic composition at different incubation conditions, including before incubation (i.e., pre-incubation), after one hour of incubation, and after two hours of incubation. The data generally showed that the pre-incubated composition had a higher pH of 6.7 than the incubated compositions, which had a pH of 6.5 or less. The pre-incubated composition also had a significantly lower amount of lactate (1.98 mmol/L) than the amount of lactate (8.04 mmol/L or higher) in the incubated compositions.

The exemplary hemostatic composition (having a low pH of 5.43) described above was subsequently tested for a platelet count (AcT Diff Counts) using an automated Coulter AcT Diff system.

TABLE 4
pH Maintenance at Pre- and Post-Lyophilization

| | AcT Diff Counts (10³/μL) |
|---|---|
| Baked, Post- | 116 |
| Lyophilization | 122 |
| Composition | 112 |
| Pre-lyophilization | 1672 |
| | 1358 |
| | 1222 |

Table 4 shows that the platelet count of the baked composition was significantly lower than the pre-lyophilization composition. A post-lyophilization heating process (referred to as “baked” composition) is performed on the dry product at 80° C. for 24 hours. At low pH, single platelets are not obtained after rehydration.

### Example 4

### pH Control and Bag Size and Fill Amounts

An exemplary hemostatic composition (StablePlate Rx®) was tested using different types of incubation bag configurations, e.g., incubation bags having different volumes and fill amounts.

The composition was tested for platelet (AcT) and flow counts after being incubated in two different test groups (Sublot A and Sublot B) using different incubation bags and fill amounts. The incubation bags were commercially available gas-permeable bags (Saint-Gobain VueLife “C” Series Bags manufactured by Saint Gobain) having fill volume sizes of 290 ml and 790 ml.

In Sublot A, an amount of 290 ml of the pre-incubated composition was added into an incubation bag having a fill volume limit of 290 ml (“290-ml bag”). During processing, the pH of Sublot A was readjusted before lyophilization occurred by adding NaOH as part of a 50/50 mixture of 1M NaOH and a loading buffer.

In Sublot B, an amount of 370 ml of pre-incubated composition was added to an incubation bag having a fill volume limit of 790 ml (“790-ml bag”) and. By filling the larger 750-ml bag at only approximately half its fill capacity, the ratio of surface area of the incubation bag relative to the volume of the composition (“SA/V ratio”) of Sublot B was greater than the SA/V ratio of Sublot A.

Lyophilization Platelet Count and pH in Bags

| | Pre- | | | | Pre- |
|---|---|---|---|---|---|
| | incuba- | Pre- | Post- | | lyophi- |
| | tion | incuba- | incuba- | Adjust- | lization |
| | sAcT Count | tion | tion | ed | AcT Count |
| Sublot | (×10³/μL) | pH | pH | pH | (×10³/μL) |
| A - 290 ml in | 2420 | 6.83 | 5.47 | 6.58 | 1613 |
| 290 cc bag | | | | | |
| B - 372 ml in | | | 6.6 | — | 2003 |
| 750 cc bag | | | | | |

Table 5 provides a summary of the platelet (AcT) count and pH data of Sublots A and B at pre-incubation, post-incubation (pH), and pre-lyophilization stages of processing. The data shows that Sublot B, which had a higher SA/V ratio, yielded a higher particle (AcT) count than Sublot A at the post-incubation and pre-lyophilization stages.

Sublots A and B were lyophilized for 2 hours at a temperature of 37 C.

TABLE 6
Post-Lyophilization Platelet Count

| | | Post-lyophilization |
|---|---|---|
| | Sublot | AcT Count × 10³/μL |
| Baked | A - 290 ml in 290 cc bag | 1685 |
| Baked | B - 372 ml in 750 cc bag | 2116 |

Table 6 provides the platelet (AcT) count of a pooled 2× vial amount of Sublots A and B following a lyophilization process and a post-lyophilization heating process (referred to as “baked” samples). Baking is performed on the dry product at 80° C. for 24 hours. The results showed that Sublot A had a lower particle (AcT) count than Sublot B. Without being bound by theory, it has been suggested that pH is correlated to gas exchange, thus augmenting the SA/V ratio of a product undergoing incubation in a gas-permeable bag would influence the pH maintenance of the composition during incubation. Accordingly, maximizing the SA/V ratio of the bag appears to improve the maintenance of the pH, which then yields higher post-hydration particle counts.

### Example 5

### Incubation Container Type

An exemplary hemostatic composition was incubated using different types of containers, e.g., gas-permeable bags and bottles. The composition was tested for platelet (AcT) and flow counts after being incubated in two different groups (Group X and Group Y).

In Group X, the composition was added to an incubation bottle and the bottle was sealed during incubation.

In Group Y, the composition was placed into an incubation bag (Saint-Gobain VueLife “C” Series Bags manufactured by Saint Gobain) having a fill volume limit.

Groups X and Y were incubated at a temperature of 37C for one-hour and two-hour time intervals. Groups X and Y were tested for pH and lactate levels prior to incubation, and after one-hour and two-hour incubation periods before the composition was lyophilization.

TABLE 7
Pre-Lyophilization pH in Bag and Bottle

| | Process Stage | pHiStat | pH meter | Lactacte |
|---|---|---|---|---|
| Group X | Pre-incubation | 6.696 | — | 1.84 |
| (Bottle) | 1 hr incubation | <6.5 | 6.47 | 5.67 |
| | 2 hr incubation | <6.5 | 5.72 | 10.06 |
| Group X | Pre-incubation | 6.87 | — | 1.85 |
| (Bottle) | 1 hr incubation | <6.5 | 6.69 | 4.22 |
| | 2 hr incubation | <6.5 | 6.56 | 6.14 |

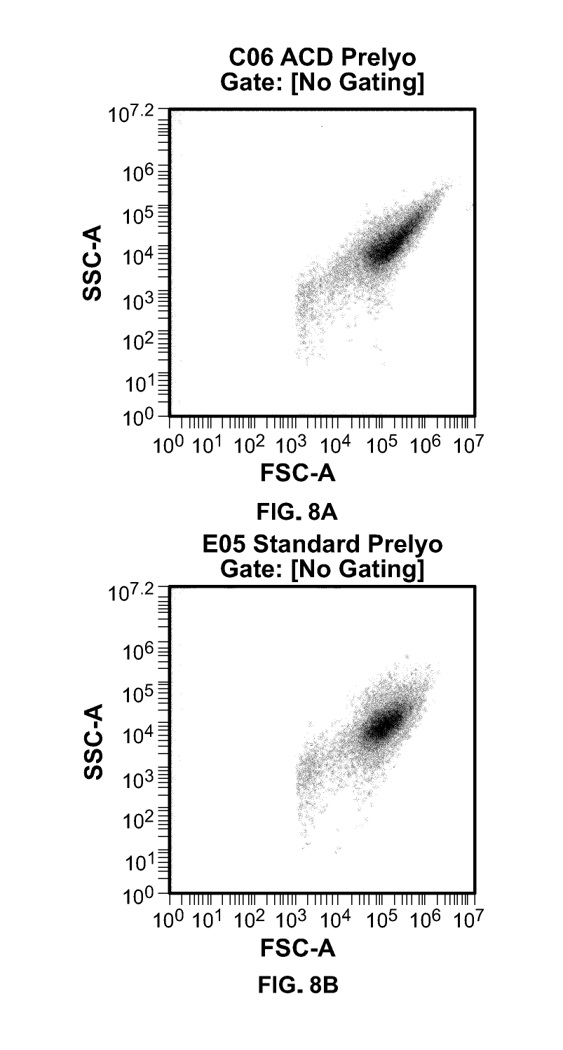

Table 7 provides the pH and lactate levels at pre-incubation, 1-hr incubation, and 2-hr incubation stages of the pre-lyophilization exemplary composition contained in a bottle (Group X) or a bag (Group Y). Table 7 and FIGS. 8A and 8B provide data showing that the composition incubated in the bag (Group Y), shown in FIG. 8A, generally maintained a higher pH level than the composition incubated in the bottle (Group X), shown in FIG. 8B, at both pre-incubation and post-incubation stages of processing. FIGS. 8A and 8B provide dot plots of flow cytometry data for Groups X and Y that showed a particle size shift in post-incubation platelets at two different pH levels (5.7 for Group X, 6.6 for Group Y).

Additionally, there were lower amounts of lactate in the composition incubated in the bag (Group Y) detected in the composition incubated in the bottle (Group X) at both pre-incubation and post-incubation stages of processing.

The compositions in Groups X and Y were lyophilized and rehydrated.

TABLE 8
Post-Lyophilization, Post-Hydrated
Platelet Count in Bag and Bottle

| Post-Hydration Product | AcT Diff Counts (*10³/μL) |
|---|---|
| Group X (Bottle) | 63 |
| Group Y (Bag) | 1752 |

Table 8 provides the platelet (AcT) count of the composition in Groups X and Y post-lyophilization and post-hydration. The data showed that the particle count of the composition incubated in the bottle (Group X) was significantly less than the particle count of the composition incubated in a bag (Group Y). These results suggest that incubation of the composition in a gas-permeable bag yields better recovery of particle counts and flow cytometry scatter profiles at the post-lyophilization and post-hydration stages. A higher particle count is indicative of a higher level of free single platelets.

### Example 6

### Bleeding Assessment in a Canine Clinical Study

A study of evaluating an exemplary lyophilized hemostatic composition in canine on-pump coronary artery bypass surgery (CABG) models was conducted. This study has applied three dose levels of the composition (5.11×10⁸, 1.57×10⁹, 5.1×10⁹particles per kg or 1,020, 3,140, or 10,200 TPU per kg, respectively). The safety and efficacy of the composition were assessed through the collection of blood loss, evaluation of blood flow through the bypass graft, evaluation of the development of acute thrombosis, and maintenance of patency through the graft over a 4-hour evaluation

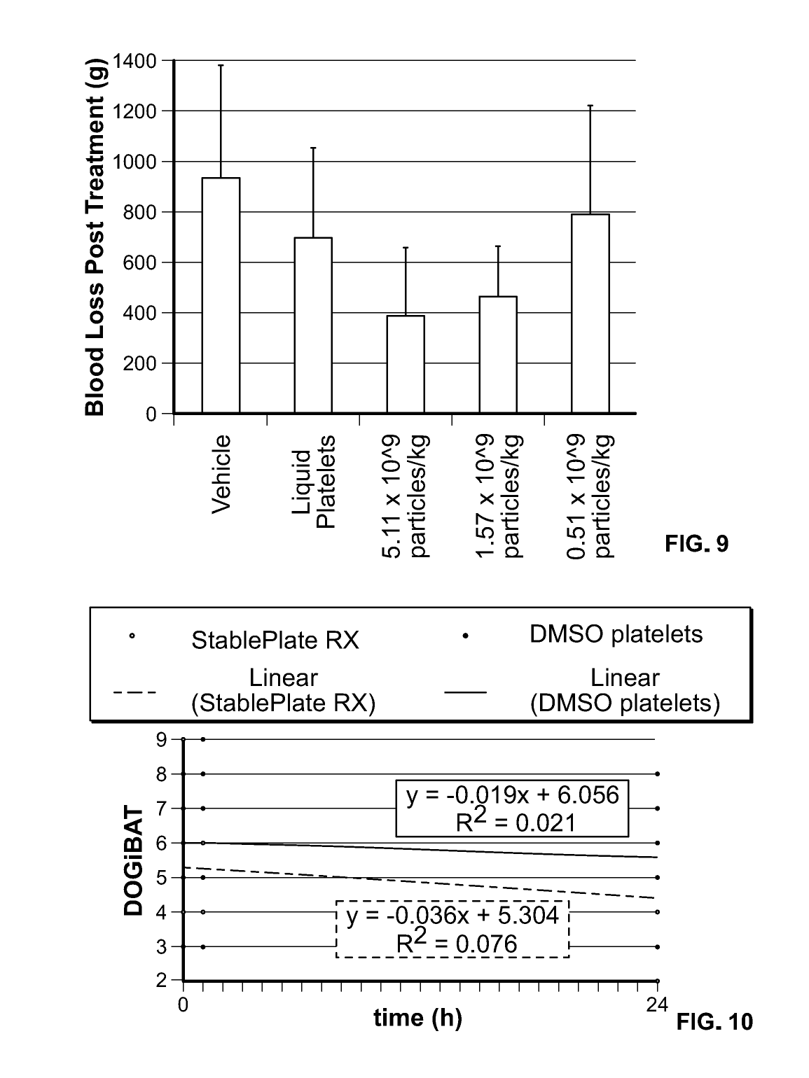

FIG. 9 provides the blood loss average (in units of gms/kg of subject body weight) of vehicle (control), fresh liquid platelets (“liquid plts”), and the three different dose levels of the composition (5.11×10⁸, 1.57×10⁹, 5.1×10⁹ particles per kg). The data showed that the exemplary composition in dosings of 1.6×10⁹ particles/kg and 5.1×10⁹ particles/kg reduced blood overall blood loss comparably to the fresh liquid platelets.

### Example 7

### Bleeding Assessment in a Clinical Study

This study evaluated an exemplary lyophilized hemostatic composition against DMSO cryopreserved platelets in controlling life threatening bleeding secondary to thrombocytopenia in canine patients. This study applied a known standardized bleeding assessment tool (DOGiBAT), as described by Makielski, to compare efficacy of the exemplary hemostatic composition (StablePlate Rx®) against the DMSO cryopreserved platelets. This study, which is still on-going, has evaluated 20 of 100 patients to date.

FIG. 10 provides initial DOGiBAT clinical data collected over a 24-hour period in which the exemplary hemostatic composition (StablePlate Rx®) or the DMSO cryopreserved platelets were used on the patients. The initial results showed that use of the exemplary hemostatic composition reduced bleeding (DOGiBAT) in patients as compared to the use of the DMSO cryopreserved platelets over the 24-hour period.

It will be apparent to those skilled in the art that various modifications and variations can be made in the practice of the present disclosure without departing from the scope or spirit of the disclosure. Other embodiments of this disclosure will be apparent to those skilled in the art from consideration of the specification and practice of the invention. It is intended that the specification and Examples be considered as exemplary only, with a true scope and spirit of the disclosure being indicated by the following claims. All references cited herein are incorporated herein by reference in their entireties.

### REFERENCES

Wilkerson, M. J. et al, Veterinary Clinical Pathology, Vol. 30, No. 3, 2001.
Makielski, K. M. et al, Development and implementation of a novel immune thrombocytopenia bleeding score for dogs, J. Vet. Intern. Med., Vol. 32, No. 3, 2018.

1. A hemostatic composition derived from canine platelets, wherein the composition does not comprise DMSO and does not comprise platelets that are chemically cross-linked by way of platelet proteins or carbohydrates found on platelet membranes.

2. The hemostatic composition of claim 1, which is in dry form, having less than ten percent moisture content.

3. The hemostatic composition of claim 2, having less than two percent moisture content.

4. The hemostatic composition of claim 1, which comprises canine platelets, particles of canine platelets, or a combination of the two.

5. The hemostatic composition of claim 1, wherein the composition comprises one or more additional blood components.

6. The hemostatic composition of claim 5, wherein the composition has less than two Endotoxin Units per milliliter.

7. The hemostatic composition of claim 1 or 5, wherein the pH of the composition is greater than 5.0.

8. The hemostatic composition of claim 7, wherein the pH of the composition is above 5.5.

9. The hemostatic composition of claim 8, wherein the pH of the composition is in a range of 6.4 to 7.4.

10. The hemostatic composition of claim 1, wherein the composition does not show observable reactivity to a human clone of an antibody that binds to CD42b when assayed by fluorescence.

11. The hemostatic composition of claim 1, wherein the composition shows observable reactivity to a human antibody that binds to CD41, a human antibody that binds to CD61, and a human antibody that binds to CD9, when assayed by fluorescence.

12. The hemostatic composition of claim 1, wherein the composition has 50% or more of particles in the range of 0.5 μm to 0.9 μm.

13. The hemostatic composition of claim 1, which is in liquid form and has 50% or more of particles in the range of 0.5 μm to 0.9 μm.

14. The hemostatic composition of claim 1, wherein the composition is stable for at least six months at temperatures that range from −20° C. to 90° C.

15. A process of making the hemostatic composition of claim 1, said process comprising:
obtaining a liquid composition that comprises canine platelets;
incubating the platelets in a solution that includes a cryoprotectant for a sufficient amount of time and at an adequate temperature to allow for entry of the cryoprotectant in to the platelets;
adding a lyoprotectant to form a drying mixture; and
drying the mixture, wherein the process includes monitoring the pH and, if necessary, adjusting the pH to maintain it above 5.0.

16. The process of claim 15, wherein the pH is maintained above 5.5.

17. The process of claim 16, wherein the pH is maintained in the range of 6.4 to 7.4.

18. The process of claim 15, wherein the liquid composition is placed a gas-permeable container.

19. The process of claim 18, wherein the gas-permeable container is a gas-permeable bag.

20. The process of claim 19, wherein the liquid composition is in the gas-permeable bag during the incubating, during the drying, or both.

21. The process of claim 18, wherein the liquid composition is placed in the gas-permeable container such that a ratio of the surface area of the gas-permeable container relative to the volume of the liquid composition contained in the gas-permeable container (“SA/V ratio”) is at least about 2.0 mL/cm².

22. The process of claim 21, wherein the liquid composition is placed in the gas-permeable container such that the SA/V ratio is at least about 3.0 mL/cm².

23. The process of claim 22, wherein the liquid composition is placed in the gas-permeable container such that the SA/V ratio is at least about 4.0 mL/cm².

24. The process of claim 23, wherein the liquid composition is placed in the gas-permeable container such that the SA/V ratio is at least about 5.0 mL/cm².

25. The process of claim 21, wherein the liquid composition is placed in the gas-permeable container such that the SAN ratio is at most 10 mL/cm².

26. The process of claim 21, wherein the liquid composition is placed in the gas-permeable container such that the SAN ratio is from about 2 mL/cm² to about 10 mL/cm².

27. The process of claim 26, wherein the liquid composition is placed in the gas-permeable container such that the SAN ratio is from about 3 mL/cm² to about 6 mL/cm².

28. The process of claim 15, wherein the adjusting the pH comprises adding NaOH to the liquid composition.

29. The process of claim 15, wherein the process does not cause aggregation of the platelets to occur.

30. A method of treating a subject experiencing bleeding, said method comprising:
contacting a site of bleeding with a sufficient amount of a composition of claim 1 to reduce or stop the bleeding.

31. The method of claim 30, wherein the step of contacting is by way of systemic administration of the composition via intravenous infusion or bolus injection.

32. The method of claim 30, wherein the step of contacting is by way of topical administration directly to the site of bleeding. For intravenous administration, the composition is a liquid composition.

33. The method of claim 30, wherein the bleeding is due to a wound or other trauma.

34. The method of claim 30, wherein the bleeding is due to coagulopathy.

35. A hemostatic composition derived from canine platelets, wherein the composition comprises less than 6 wt. % DMSO and comprises 50% or more of particles in the range of 0.5 μm to 0.9 μm.

36. The hemostatic composition of claim 35, wherein the pH of the composition is greater than 5.0.

37. The hemostatic composition of claim 36, wherein the pH of the composition is above 5.5.

38. The hemostatic composition of claim 36, wherein the pH of the composition is in a range of 6.4 to 7.4.

39. The hemostatic composition of any one of claims 35 to 38, wherein the composition does not show observable reactivity to a human clone of an antibody that binds to CD42b when assayed by fluorescence.

40. The hemostatic composition of any one of claims 35 to 39, wherein the composition shows observable reactivity to a human antibody that binds to CD41, a human antibody that binds to CD61, and a human antibody that binds to CD9, when assayed by fluorescence.

41. The hemostatic composition of any one of claims 35 to 40, wherein the composition has 50% or more of particles in the range of 0.5 μm to 0.9 μm.

42. The hemostatic composition of any one of claims 35 to 41, wherein the composition is stable for at least six months at temperatures that range from −20° C. to 90° C.

43. A hemostatic composition obtained by a process comprising the steps of:
providing a composition comprising canine platelets in a gas-permeable container;
adding a cryoprotectant to the composition;
incubating the canine platelets in the composition;
adding a lyoprotectant to the composition; and
drying the composition;
wherein the pH of the composition during the incubating, the drying, or both, is greater than 5.0.

44. The composition of claim 43, wherein the pH of the composition is greater than 5.3.

45. The composition of claim 44, wherein the pH of the composition is greater than 5.5.

46. The composition of claim 45, wherein the pH of the composition is greater than 6.0.

47. The composition of claim 46, wherein the pH of the composition is in a range of about 5.0 to about 7.4.

48. The composition of claim 47, wherein the pH of the composition is in a range of about 5.5 to about 7.4.

49. The composition of claim 48, wherein the pH of the composition is in a range of about 6.4 to about 7.4.

50. The composition of any one of claims 43 to 46, wherein the pH of the composition is lower than 10.0.

51. The composition of any one of claims 43 to 46, wherein the pH of the composition is lower than about 9.0.

52. The composition of any one of claims 43 to 46, wherein the pH of the composition is lower than about 8.0.

53. The composition of any one of claims 43 to 46, wherein the pH of the composition is lower than about 7.5.

54. The composition of any one of claims 47 to 53, wherein the pH of the composition does not increase or decrease more than 2.4.

55. The composition of claim 54, wherein the pH of the composition does not increase or decrease more than 2.0.

56. The composition of claim 55, wherein the pH of the composition does not increase or decrease more than 1.5.

57. The composition of claim 56, wherein the pH of the composition does not increase or decrease more than 1.0.

58. The hemostatic composition of claim 1, wherein the composition has 50% or more of particles in the range of 0.5 μm to 0.9 μm and the remaining particles in the range of 0.9 μm to 2.5 μm.

59. The hemostatic composition of claim 1, which is in liquid form and has 50% or more of particles in the range of 0.5 μm to 0.9 μm and the remaining particles in the range of 0.9 μm to 2.5 μm.

60. A process for making a hemostatic composition, said process comprising:
incubating a liquid composition that comprises canine platelets in a solution that includes a cryoprotectant;
adding a lyoprotectant to form a mixture; and
drying the mixture;
wherein the process includes maintaining the pH above 5.

61. The process of claim 60, wherein the pH of the composition is greater than 5.3.

62. The process of claim 61, wherein the pH of the composition is greater than 5.5.

63. The process of claim 62, wherein the pH of the composition is greater than 6.0.

64. The process of claim 63, wherein the pH of the composition is in a range of about 5.0 to about 7.4.

65. The process of claim 64, wherein the pH of the composition is in a range of about 5.5 to about 7.4.

66. The process of claim 61, wherein the pH of the composition is in a range of about 6.4 to about 7.65.

67. The process of any one of claims 60 to 66, wherein the pH of the composition is lower than about 10.0.

68. The process of any one of claims 60 to 66, wherein the pH of the composition is lower than about 9.0.

69. The process of any one of claims 60 to 66, wherein the pH of the composition is lower than about 8.0.

70. The process of any one of claims 60 to 66, wherein the pH of the composition is lower than 7.5.

71. The process of any one of claims 60 to 66, wherein the pH of the composition does not increase or decrease more than 2.4.

72. The process of claim 71, wherein the pH of the composition does not increase or decrease more than 2.0.

73. The process of claim 72, wherein the pH of the composition does not increase or decrease more than 1.5.

74. The process of claim 73, wherein the pH of the composition does not increase or decrease more than 1.0.

75. The process of claim 60, wherein the incubating is carried out for about 1 minute to about 180 minutes.

76. The process of claim 75, wherein the incubating is carried out for about 110 minute to about 130 minutes.

77. The process of claim 60, wherein the incubating is carried out at a temperature above freezing.

78. The process of claim 77, wherein the incubating is performed at a temperature of about 20° C. to about 42° C.

79. The process of claim 78, wherein the incubating is performed at a temperature of about 35° C. to about 40° C.
